(19) United States
(12) Patent Application Publication
(10) Pub. No.: US 2013/0260411 A1
(43) Pub. Date: Oct. 3, 2013

(54) Modular compositing screening protocols

(71) Applicant: Mansour Samadpour, Lake Forest Park, WA (US)

(72) Inventor: Mansour Samadpour, Lake Forest Park, WA (US)

(21) Appl. No.: 12/927,849

(22) Filed: Nov. 26, 2010

(51) Int. Cl. C12Q 1/04

(52) U.S. Cl. CPC C12Q 1/04
USPC 435/34

(57) ABSTRACT

Multiple samples are taken from multiple units of food product to be tested for pathogen or other microbes, with the samples being pooled for composite testing, individual testing being required only in the event that the pool indicates positive.

The disclosure of U.S. Pat. No. 7,531,163 is incorporated by reference herein as background material. This-application provides additional inventive examples and methods in the same field of application. The examples and methods are as follows.

### BRIEF DESCRIPTION OF THE DRAWINGS

FIG. 1 is a flow diagram illustrating the first example embodiment of the the invention.

FIG. 2 is a flow diagram illustrating the second example.

FIG. 3 is a flow diagram that illustrates the third example.

FIG. 4 is a flow diagram that illustrates the fourth example.

FIG. 5 is a flow diagram that illustrates the fifth example.

FIG. 6 is a flow diagram that illustrates the sixth example.

FIG. 7 is a flow diagram that illustrates the seventh example.

FIG. 8 is a flow diagram that illustrates the eighth example.

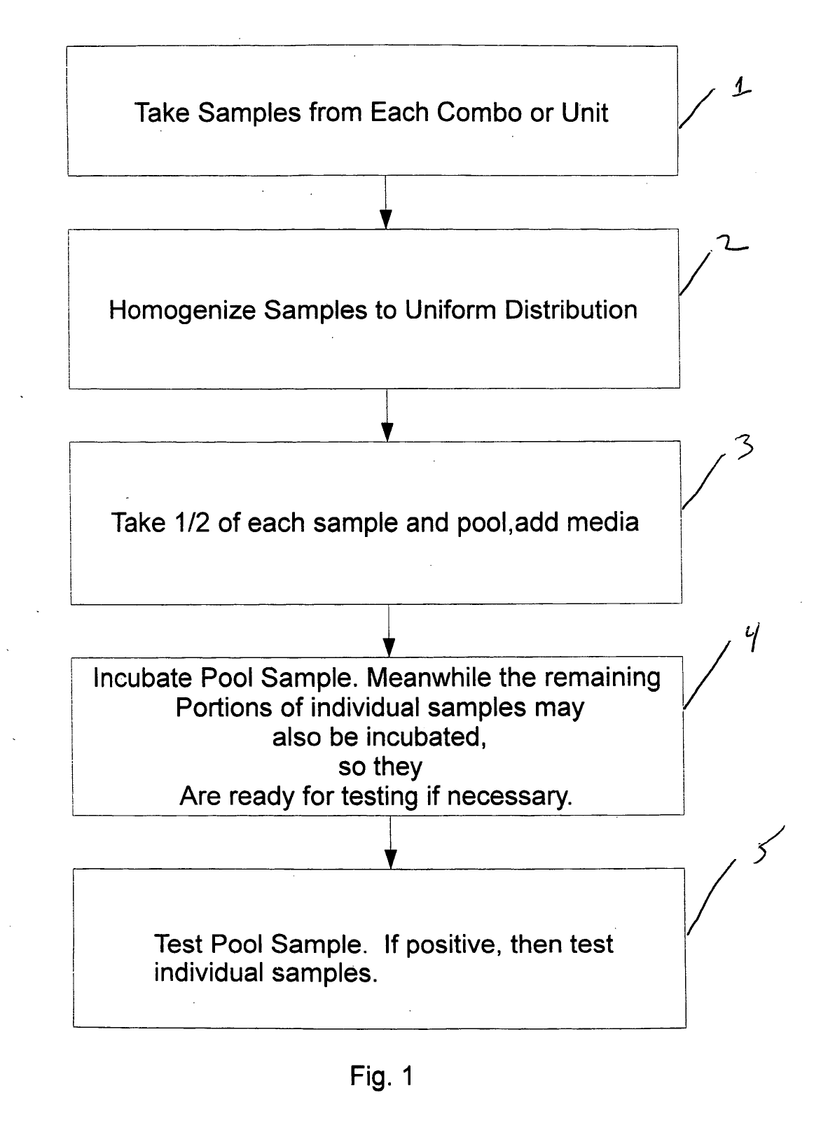

FIG. 1 is a flow chart that illustrates the first example. The steps for this example are as follows:

(1) Collect twice as much sample (e.g. N=120 instead of N=60, or portions twice as big)

a. Homogenize to uniform distribution

b. Pool ½ portions of all samples (matrix+media)

c. Incubate pool sample+individual samples until CFU>MDL

d. Test pool
i. If negative, release
ii. If positive, test individual samples by same method or alternative method

Referring to FIG. 1, in step 1, sample is collected from individual units of product such as beef combos. More samples are preferably collected in this method because the samples will be split prior to enrichment, so the chances of having similar microbe populations in the split samples are better if a larger number of samples are taken. A larger number of samples is preferably, but not strictly necessary, and the method would work with the normal number of samples, with the caveat that it might be somewhat less accurate when microbe populations are very sparse. In step 2, the samples are homogenized to uniform distribution. In step 3, a portion of each sample is taken and placed into a pool

FIG. 1 states that the portion is ½. This is a convenient portion, but other portions will also work. In step 4, the pooled samples are incubated to increase the population of microbes to detectable levels, as necessary. Preferably, the remaining portions of individual samples that were split off can also be incubated at this time so that they are ready for testing if need be. In step 5, the pool is tested. If the test shows positive, then testing of the retained portions of individual samples can be done in order to determine the source of the contamination.

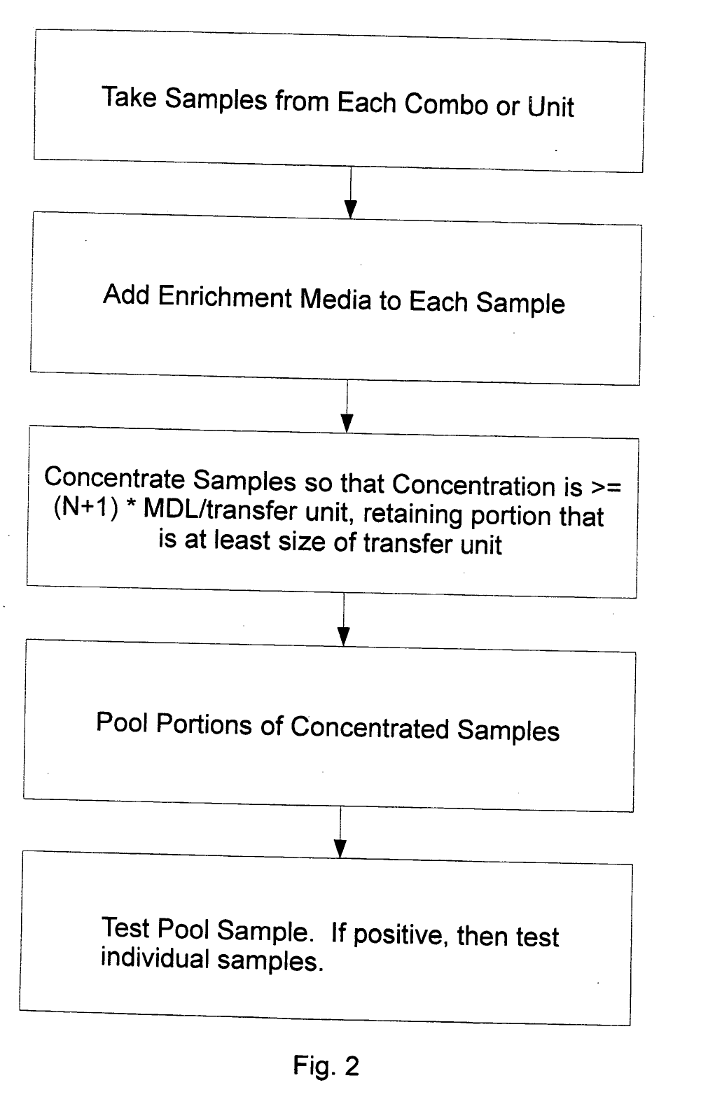

FIG. 2 is a flow chart that illustrates the second example. The steps for this example are as follows:

(2) Add enrichment media to each sample.

a. Concentrate sample so that CFU of analysis target in concentrated form is present at (N+1)×MDL/transfer unit, where N=number of contributors to a pool and transfer unit is the portion of concentrated form of sample that will be contributed to a pooled sample. Methods to concentrate sample include, but are not limited to:
i. Affinity chromatography Immunomagnetic beads
iii. Centrifugation followed by recovery of pellet of viable organisms

b. Pool portions of concentrated forms of sample, retaining a portion that is at least of size transfer unit/N (so that retained portion, transfer unit/N×(N+1)×MDL/transfer unit contains (N+1)N×MDL of target organism.

c. Test pool
i. If negative, release
ii. If positive, test individual samples by same method or alternative method
   In the foregoing, the statements concerning required concentration of analysis target and the size of the transfer unit and pool are statements of preference. Variations from these preferences are possible within the spirit of the invention.

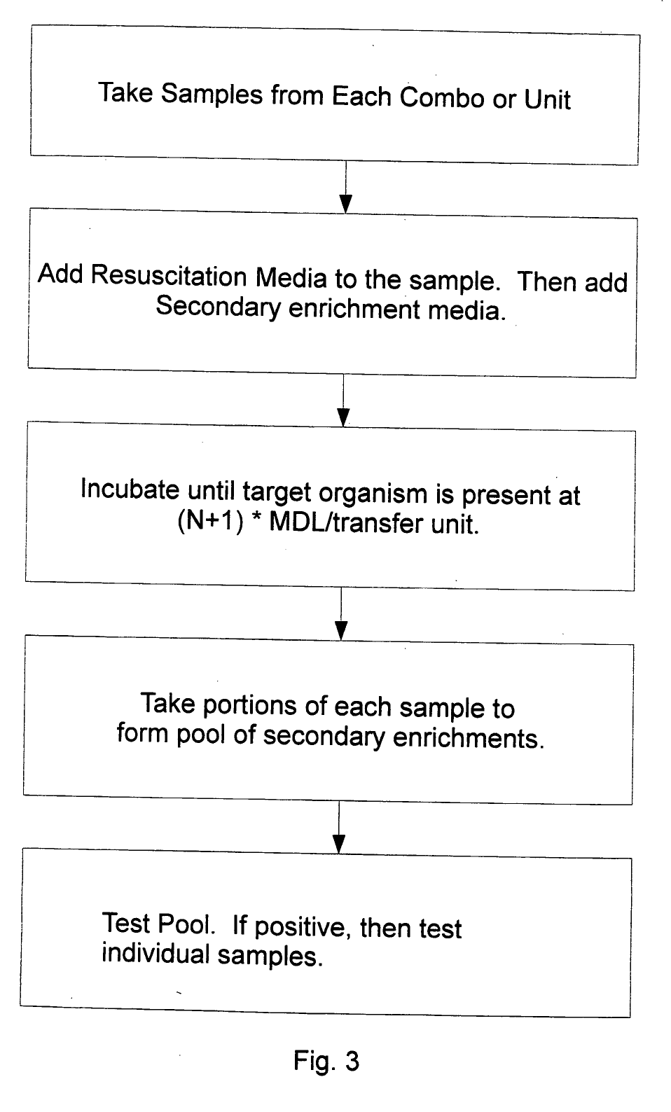

FIG. 3 is a flow chart that illustrates the third example. The steps for this example are as follows:

(3) Add resuscitation media to samples

a. After appropriate period of time add secondary enrichment media to same sample bag.

b. Incubate combined resuscitation/secondary enrichments until target organism is present at (N+1)×MDL/transfer unit, where N=number of contributors to a pool.

c. Form pool of N transfer units of secondary enrichments

d. Test pool
i. If negative, release
ii. If positive, test individual samples by same method or alternative method

In the foregoing, the statements concerning required concentration of analysis target and the size of the transfer unit and pool are statements of preference. Variations from these preferences are possible within the spirit of the invention.

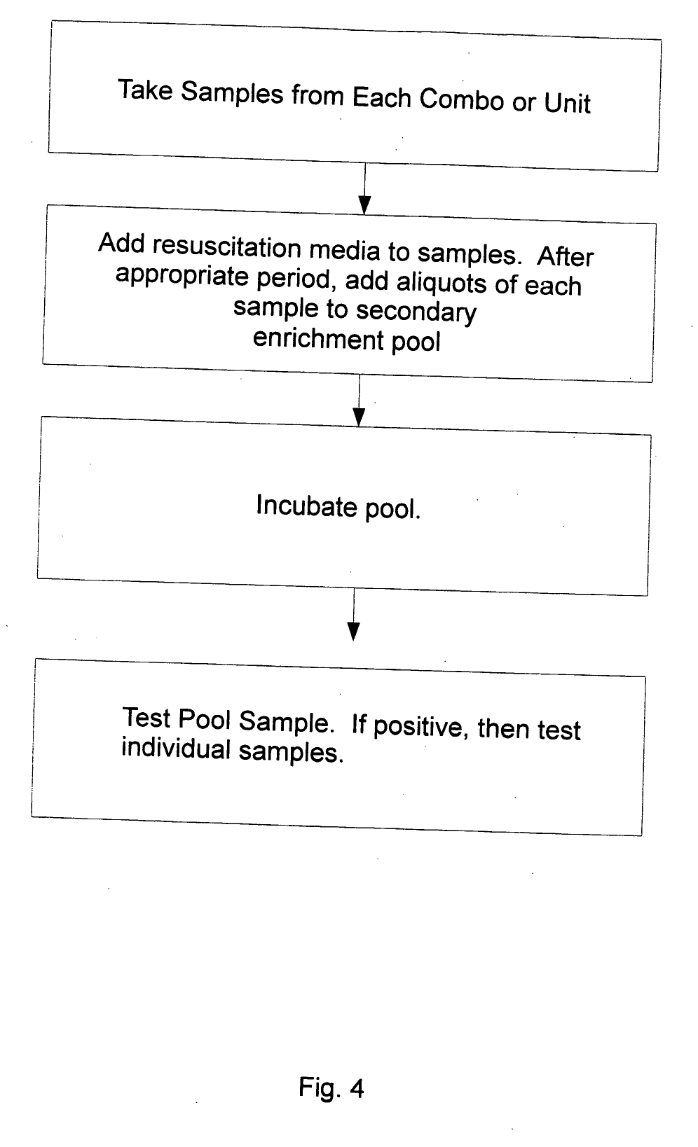

FIG. 4 is a flow chart that illustrates the fourth example. The steps for this example are as follows:

(4) Add resuscitation media to samples

a. After appropriate period of time transfer aliquots from resuscitation media to secondary enrichment pool. Should have>1 CFU/transfer unit.

b. Incubate pool until CFU>MDL

c. Test pool
i. If negative, release
ii. If positive, transfer aliquots from resuscitation media to individual secondary enrichments. Should have>1 CFU/transfer unit. Incubate individual secondary enrichments until CFU>MDL. Test by same method or alternative method.

In the foregoing, the statements concerning required concentration of analysis target and the size of the transfer unit are statements of preference. Variations from these preferences are possible within the spirit of the invention.

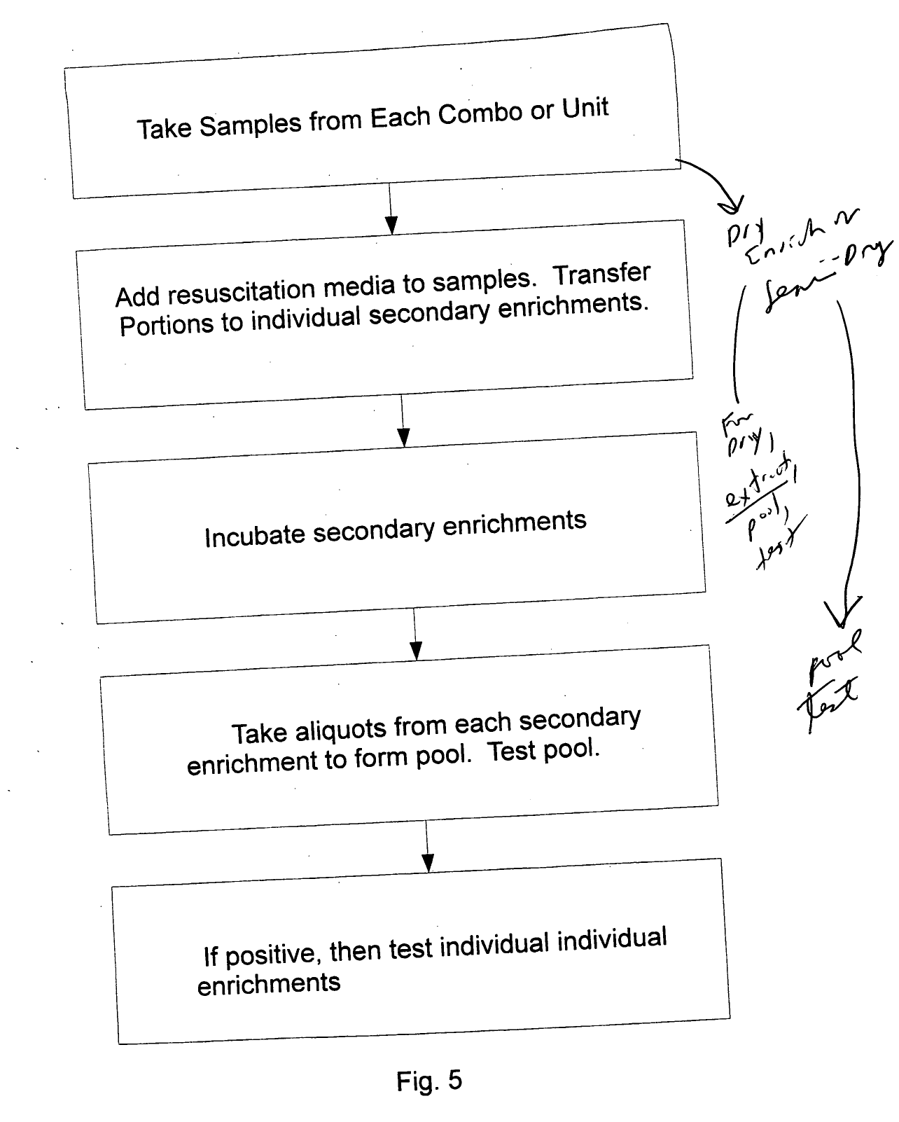

FIG. 5 is a flow chart that illustrates the fifth example. The steps for this example are as follows:

(5) Add resuscitation media to samples

a. Transfer an aliquot from resuscitation media to individual secondary enrichments. Should have>1 CFU/transfer unit.

b. Incubate secondary enrichments until target organism is present at (N+1)×MDL, where N=number of contributors to a pool.

c. Form pool of N transfer units of secondary enrichments

d. Test pool
i. If negative, release
ii. If positive, test individual samples by same method or alternative method

In the foregoing, the statements concerning required concentration of analysis target and the size of the transfer unit are statements of preference. Variations from these preferences are possible within the spirit of the invention.

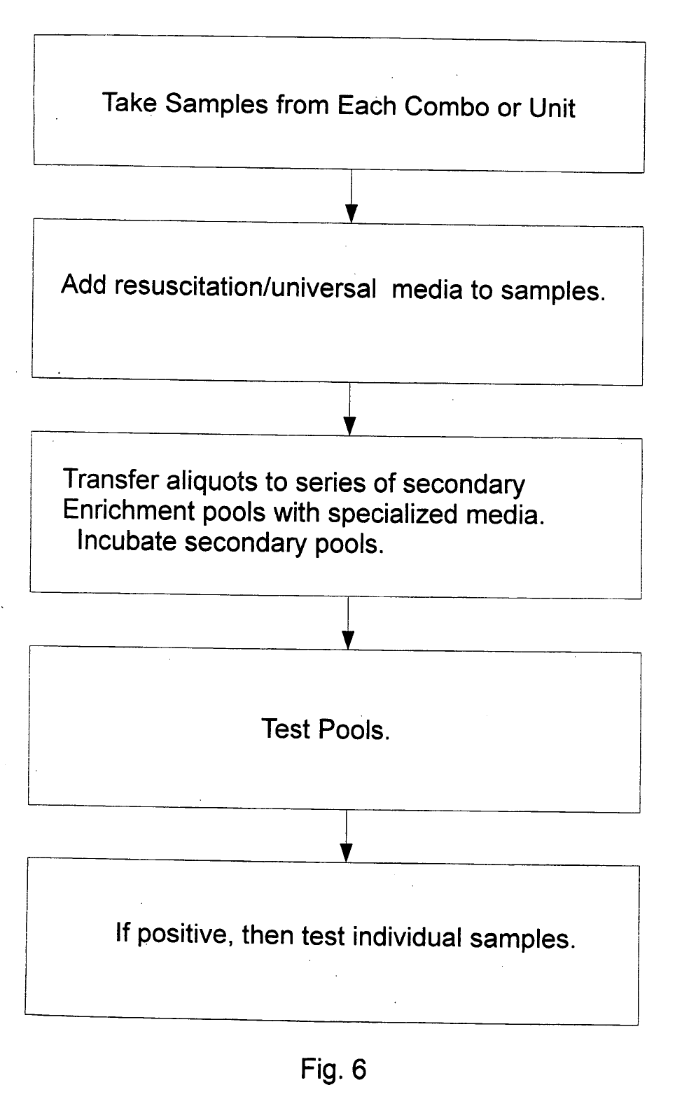

FIG. 6 is a flow chart that illustrates the sixth example. The steps for this example are as follows:

(6) Add resuscitation/universal media to samples

a. Incubate for appropriate period of time. Considering an array of possible analysis targets, enrich until the slowest growing target is present at>1 CFU/transfer unit.

b. Considering an array of possible analysis targets, transfer aliquots from resuscitation/universal media to a series of secondary enrichment pools where each individual enrichment pool is composed of a specialized media selected to promote a specific analysis target or a group of analysis targets.

c. Incubate individual secondary enrichment pools as appropriate until CFU>MDL for analysis target associated with that individual pool.

d. Test pool
i. If negative, release
ii. If positive, test individual samples by same method or alternative method

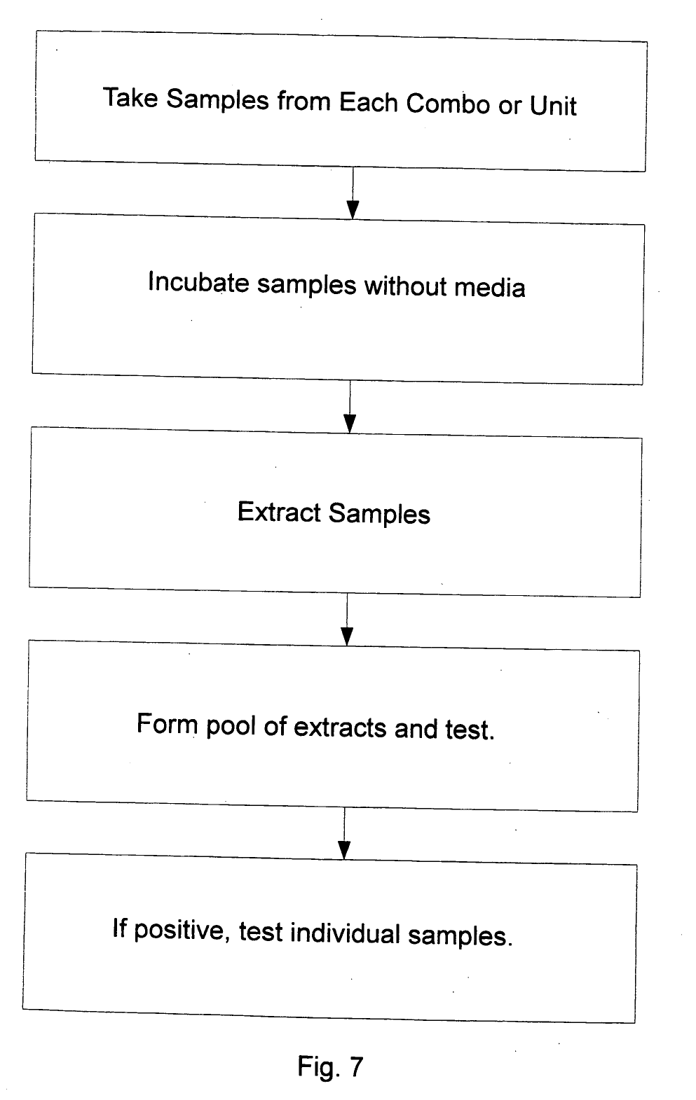

FIG. 7 is a flow chart that illustrates the seventh example. The steps for this example are as follows:

(7) Incubate samples without media (dry enrichment as taught in U.S. Pat. No. 7,531,163)

a. Applicable to samples where nature of sample provides nutrition to allow analysis target organisms to proliferate to point where they are present at (N+1)×MDL×(extraction media volume/transfer unit), where N=number of contributors to a pool, extraction media is explained below, and transfer unit is aliquot removed to form pool.

b. Extract samples by adding and recovering an appropriate liquid media to a ‘recovered sample’
i. Enrichment
Neutral (e.g. peptone water)
iii. In rare cases, could be lysis, provided that non-target cells are not present in excessive numbers (e.g. something grows on sugar as ‘dry enrichment’)

c. Form pool of N transfer units of ‘recovered samples’

d. Test pool
i. If negative, release
ii. If positive, test individual samples by same method or alternative method in the foregoing, the statements concerning required concentration of analysis target and the size of the transfer unit are statements of preference. Variations from these preferences are possible within the spirit of the invention.

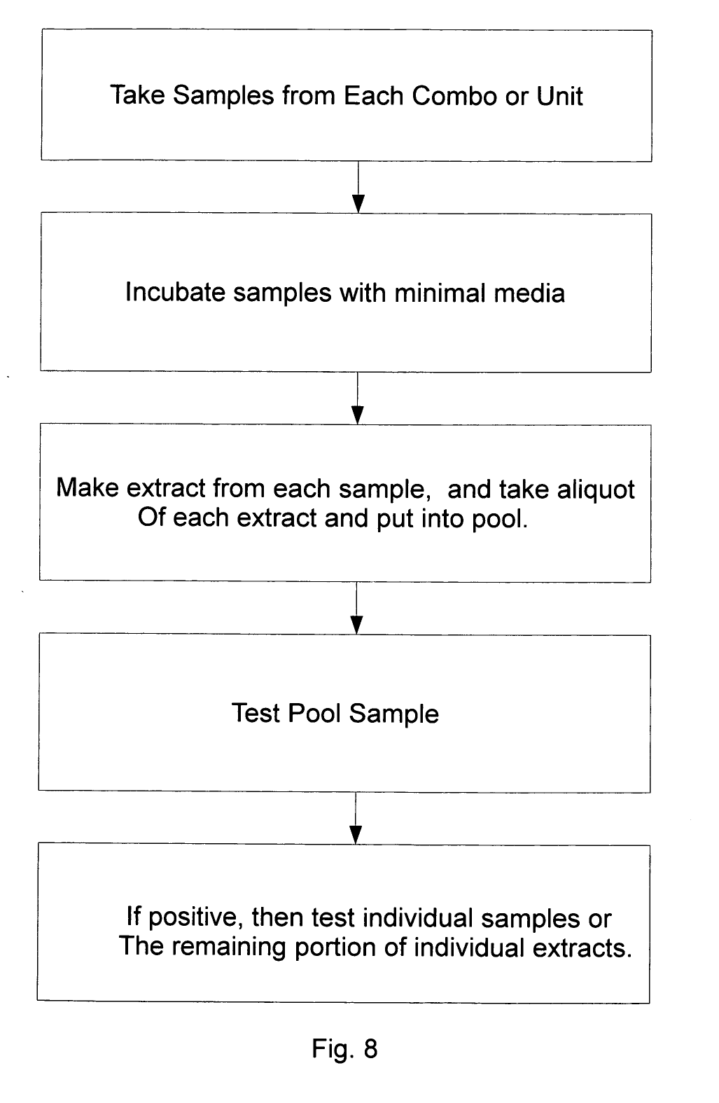

FIG. 8 is a flow chart that illustrates the 8th example. The steps for this example are as follows:

(8) Incubate samples with minimal media (semi-dry enrichments taught in U.S. Pat. No. 7,531,163)

a. Applicable to samples where nature of sample provides nutrition to allow analysis target organisms to proliferate to point where they are present at (N+1)×MDL×(semi-dry media volume/transfer unit), where N=lumber of contributors to a pool, extraction media is explained below, and transfer unit is aliquot removed to form pool.

b. Form pool of N transfer units of ‘semi-dry enrichment samples’

c. Test pool
i. If negative, release
ii. If positive, test individual samples by same method or alternative method

In the foregoing, the statements concerning required concentration of analysis target and the size of the transfer unit are statements of preference. Variations from these preferences are possible within the spirit of the invention.

1. I claim the the methods stated in the above examples.
